(19) United States
(12) Patent Application Publication
(10) Pub. No.: US 2026/0215828 A1
(43) Pub. Date: Jul. 30, 2026

(54) ORTHOPEDIC SURGICAL ALIGNMENT AND STABILIZATION TOOL

(71) Applicant: MDS II, LLC, Boulder, CO (US)

(72) Inventors: Omer Mei-Dan, Boulder, CO (US); Jacob Segil, Boulder, CO (US); Louis Piccione, Broomfield, CO (US); Sydney Levy, Berthoud, CO (US)

(21) Appl. No.: 19/148,654

(22) PCT Filed: Jan. 18, 2024
(86) PCT No.: PCT/US2024/012036

§ 371 Date: Jul. 16, 2025

Related U.S. Application Data
(60) Provisional application No. 63/440,063, filed on Jan. 19, 2023.

(51) Int. Cl. A61B 17/80; A61B 17/68

(52) U.S. Cl. CPC A61B 17/8019; A61B 2017/681

(57) ABSTRACT

Embodiments are directed to an orthopedic fixation system that includes an orthopedic plate defining multiple openings each configured to engage with an orthopedic screw to secure the orthopedic plate to a bone and a recess positioned along a side of the orthopedic plate. The fixation system can include a first pin configured to pass through an opening of the multiple openings and be secured into the bone and a second pin that is positioned at least partially within the recess when the orthopedic plate is in an unrotated configuration. The fixation system can include a rotation mechanism that includes a first body component that couples to the first pin and defines includes a guide, and a second body component that couples to the first body component and the second pin, where the second body component is configured move with respect to the first body component along the guide.

### CROSS-REFERENCE TO RELATED APPLICATION

This Patent Cooperation Treaty Patent application claims priority to U.S. Provisional Patent Application 63/440,063, filed Jan. 19, 2023, titled “Orthopedic Surgical Alignment and Stabilization Tool” the contents of which are incorporated herein by reference.

### FIELD

The described embodiments relate generally to medical devices for treating orthopedic fractures and more particularly, the present embodiments relate to fixation systems for controlling bone movement during a surgical procedure.

### BACKGROUND

Orthopedic procedures may be performed to correct or change an alignment of a user's joints. For example, some orthopedic procedures may include cutting a patient's femur and rotating a lower portion of the femur to change an alignment of the patient's lower leg (e.g., knee and ankle) with respect to an upper portion of their leg (e.g., hip). In other cases trauma can cause a bone, such as the femur, to break and become misaligned.

In the cases of reconstructive surgeries, a surgeon cuts the femur and manual manipulates the user's lower leg to a desired position. After the lower portion of the femur is in the desired orientation, the surgeon may install an orthopedic plate to fix the two sections of the femur together while the bone heals. It may be difficult to precisely position a lower portion of the femur with respect to the upper portion after it has been cut or fractured. For example, muscles and/or ligaments may pull the sections of the femur in different directions making it harder for a surgeon to precisely align the sections of the femur.

### SUMMARY

Embodiments are directed to an orthopedic fixation system that includes an orthopedic plate defining multiple openings each configured to engage with an orthopedic screw to secure the orthopedic plate to a bone and a recess positioned along a side of the orthopedic plate. The orthopedic fixation system can include a set of pins that includes a first pin configured to pass through an opening of the multiple openings and be secured into the bone and a second pin that is positioned at least partially within the recess when the orthopedic plate is fixed to the bone and be secured into the bone. The orthopedic fixation system can also include a rotation mechanism that includes a first body component that couples to the first pin and has a guide and a rotation indicator; and a second body component that couples to the first body component and the second pin. The second body component can be configured move with respect to the first body component along the guide. The guide can cause the second body component to rotate substantially about an axis of the bone and the second body component can indicates an amount of rotation on the rotation indicator.

Embodiments are directed to an orthopedic fixation system that includes a first pin configured to be secured into a bone and a second pin configured to be secured into the bone. The orthopedic fixation system can include a rotation mechanism that includes a first body component that couples to the first pin and comprises a guide and a second body component that couples to the second pin. The second body component can be configured move along the guide and the guide can cause the second body component to substantially rotate about an axis of the bone.

Embodiments include an orthopedic fixation system that includes an orthopedic plate defining an opening a recess positioned along a side of the orthopedic plate. The orthopedic fixation system can include a first pin configured to pass through the opening and be secured into a bone and a second pin configured to be secured into the bone. The orthopedic fixation system can also include a rotation mechanism that includes a first body component that couples to the first pin and comprises a guide, and a second body component that couples to the second pin. The second body component can be configured to move along the guide, and the guide can cause the second body component to substantially rotate about an axis of the bone.

### BRIEF DESCRIPTION OF THE DRAWINGS

The disclosure will be readily understood by the following detailed description in conjunction with the accompanying drawings, wherein like reference numerals designate like structural elements, and in which:

FIG. 1A shows an example orthopedic fixation system coupled to a bone and in an unrotated configuration;

FIG. 1B shows an example orthopedic fixation system coupled to a bone and in a rotated configuration;

FIG. 2 shows an example orthopedic fixation system;

FIG. 3 shows an example orthopedic fixation system with the rotation mechanism removed from the pins;

FIG. 4 shows an example of coupling of the rotation mechanism to the pins;

FIG. 5 shows an example orthopedic fixation system;

FIG. 6 shows an example rotation indicator for the orthopedic fixation system shown in FIG. 5;

FIG. 7 shows an example orthopedic fixation system including a guide;

FIG. 8 shows an example orthopedic fixation system;

FIG. 9 shows an example orthopedic plate that can be used with the orthopedic fixation systems described herein; and

FIG. 10 shows an example orthopedic fixation system including an alignment tool for use with an orthopedic plate.

It should be understood that the proportions and dimensions (either relative or absolute) of the various features and elements (and collections and groupings thereof) and the boundaries, separations, and positional relationships presented therebetween, are provided in the accompanying figures merely to facilitate an understanding of the various embodiments described herein and, accordingly, may not necessarily be presented or illustrated to scale, and are not intended to indicate any preference or requirement for an illustrated embodiment to the exclusion of embodiments described with reference thereto.

### DETAILED DESCRIPTION

Reference will now be made in detail to representative embodiments illustrated in the accompanying drawings. It should be understood that the following descriptions are not intended to limit the embodiments to one preferred embodiment. To the contrary, it is intended to cover alternatives, modifications, and equivalents as can be included within the spirit and scope of the described embodiments as defined by the appended claims.

Embodiments disclosed herein are directed to an orthopedic fixation system that can be used to control movement of a fractured bone during a surgical procedure. The orthopedic fixation system can include an orthopedic plate that is used to secure two segments of a fractured bone together, multiple pins that are secured into the bone during a surgical procedure and a rotation mechanism.

During an orthopedic procedure a bone may be cut to change alignment of different segments of a bone. For example, some procedures may cut the femur and rotate a lower portion of the femur with respect to an upper portion of the femur to change an alignment of the patient's lower leg. This type of surgical procedure may be used to decrease wear on joints, improve a patient's gait and/or for a variety of other reasons. During the procedure a surgeon typically cuts the femur and then manually re-aligns the lower section of femur and uses a plate to secure the cut sections of the femur together once the desired alignment is achieved. However, it may be difficult to precisely control the alignment of the cut portions of the femur and/or accurately measure the change in alignment.

The orthopedic fixation systems described here can be used to stabilize the different sections of the bone and control movement of the bone segments after they are cut by a surgeon or otherwise fractured. In some cases, the orthopedic fixation system may be secured to a patient's bone (e.g., femur) prior to the bone being cut/fractured. In these cases, once the bone is cut, the orthopedic fixation system may be locked to stabilize the bone so that is does not move due to muscle tension or other forces acting on the bone. The orthopedic fixation system can be unlocked and a surgeon (and/or other medical professional) can rotate the bone along a controlled path defined by the orthopedic fixation system. For example, the orthopedic fixation system may define a movement path that primarily allows rotation of the bone about an axis that is aligned along a length of the bone. Once in the desired location the surgeon may lock the orthopedic fixation system thereby preventing further rotation/movement of the bone and attach an orthopedic plate to the bone to fix the fractured bone segments together.

In some cases, the orthopedic plate can be secured to the bone prior to the bone being cut/fractured into two segments. The plate may be secured to the bone on one side of the intended fracture site using one or more pins which extend out of the patient's body and/or one or more orthopedic screws. One or more additional pins may be secured into the bone on the other side of the intended fracture site and extend out of the patient. The rotation mechanism can be secured to the pins and the bone can be cut to separate a first portion of the bone (e.g., upper femur segment) from a second portion of the bone (e.g., lower femur segment).

The rotation mechanism can be used to control movement of the bone by the surgeon. For example, the rotation mechanism may include a first component that is coupled to a first pin on the first side of the fracture and a second component that is coupled to a second pin on the second side of the fracture. The rotation mechanism may define a movement path that allows rotation of the second portion of the bone about an axis that is aligned with or close to an axis along a length of the bone. The surgeon may cause the second portion of the bone to rotate, for example, in the case of a procedure on the femur, the surgeon may grasp a user's leg and rotate their leg and lower femur segment while the upper femur segment remains stationary. The orthopedic fixation system may constrain the movement to defined movement path (e.g., rotation about an axis).

Once the second portion of the bone (e.g., lower femur portion) is aligned to the first portion of the bone (e.g., upper femur portion) in a desired orientation, orthopedic screws can be used to fix the orthopedic plate to both of the bone segments thereby fixing the bone in the realigned configuration. The rotation mechanism and pins can be removed from the bone after the plate has fixed to the bone segments.

In some cases, the orthopedic plate may include one or more recesses that allow one or more pins coupled to a second component of the rotation mechanism to rotate with respect to the plate when the second bone segment is rotated. For example, the pins may be secured to the user in an unrotated state and both the stationary pin(s) coupled to the first component of the rotation mechanism and the movable pins(s) couple to the second component of the rotation mechanism may be aligned along a length of the plate. The moving pins may be initially positioned at least partially within a recess in the orthopedic plate and move (e.g., rotate) away from the plate and out of the recess when the second bone segment is rotated.

The orthopedic fixation system may also include a rotation indicator which can indicate an amount that the second bone segment was rotated with respect to the first bone segment. For example, the first component of the rotation mechanism may include a scale and the second component of the rotation mechanism may include an indicator to moves along the scale as the second component is moved. The scale and indicator can be configured to indicate an amount of rotation of the second bone segment with respect to the first bone segment.

The orthopedic fixation system may also include a locking mechanism which can function to selectively lock the rotation mechanism in place. For example, the rotation mechanism may be locked after it is secured to the bone and prior to the bone being cut into two segments. After the cutting procedure, the locking mechanism may be unlocked allowing movement of the second bone segment with respect to the first bone segment and then re-locked when a desired orientation of the bone segments is achieved. The locking mechanism may help maintain the bone segments in the desired orientation while the orthopedic plate is secured to each of the bone segments fixing the bone in the new orientation.

The examples described herein are discussed in the context of orthopedic procedures where the bone is intentionally fractured by a surgeon for realignment. However, the orthopedic fixation system described herein can be secured to an already fractured bone (e.g., due to injury) and used to achieve a desired alignment of the fractured bone segments. The orthopedic fixation systems described herein can be used in procedures on bones in the leg (e.g., femur, tibia, and so on), arms (humerus, radius, ulna, and so on), or other bones having sufficient length.

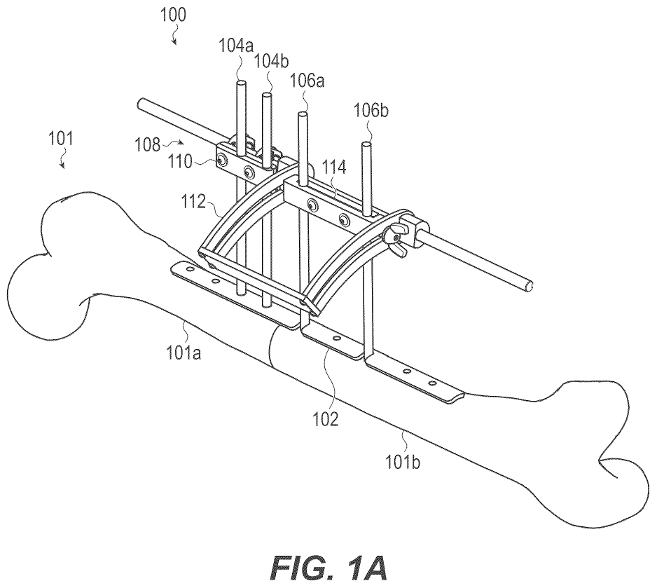

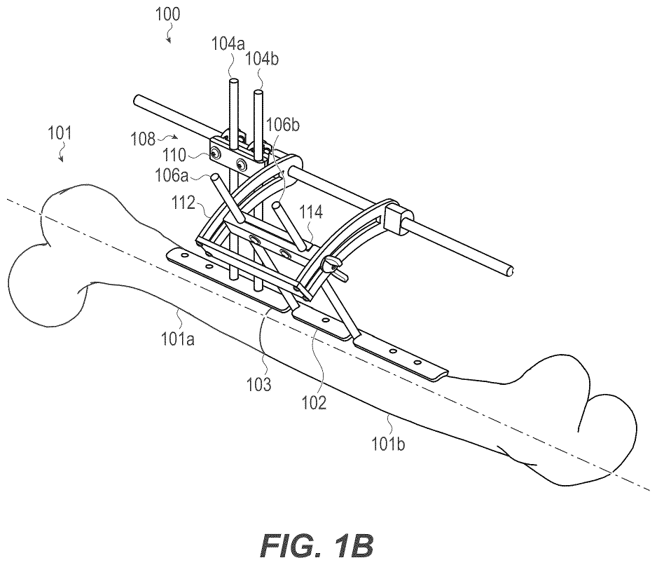

These and other embodiments are discussed below with reference to FIGS. 1-10. However, those skilled in the art will readily appreciate that the detailed description given herein with respect to these Figures is for explanatory purposes only and should not be construed as limiting.

FIG. 1A shows an example orthopedic fixation system 100 secured to a bone 101 in an unrotated configuration. The orthopedic fixation system 100 can include an orthopedic plate 102, one or more stationary pins 104, one or more movable pins 106, and a rotation mechanism 108 comprising a first body component 110 and a second body component 114. The orthopedic fixation system 100 can be secured to the bone 101 during a procedure and used to fix a first bone segment 101a and a second bone segment 101b in a fixed relation after the bone 101 is cut or fractured and control relative movement of the first bone segment 101a with respect to the second bone segment 101b, as described herein.

During a surgical procedure the orthopedic plate 102 can be coupled to the bone 101. The orthopedic plate 102 can be secured to the bone 101 prior to the bone being cut. The orthopedic plate 102 is coupled to the bone at a first side of a planned fracture location 103. For example, the orthopedic plate 102 can be coupled to the first bone segment 101a prior to cutting the bone using one or more orthopedic screws.

One or more stationary pins 104 can be coupled into the bone 101 at the first side of the planned fracture location 103 (e.g., the first bone segment 101a) prior to cutting the bone. The end of the stationary pin(s) 104 can include threads that are configured to be screwed into the bone. In other cases, the stationary pin(s) 104 can be coupled to the bone 101 using any other suitable techniques. The stationary pin(s) 104 can each pass through an opening in the orthopedic plate. The openings in the orthopedic plate may be configured to secure the orthopedic plate to the bone once the stationary pins 104 are removed using any suitable technique (e.g., orthopedic screws). In some cases, the stationary pins 104 can include features that couple the orthopedic plate 102 to the bone 101. For example, the openings in the orthopedic plate 102 may include a tapered opening and the stationary pins 104 can include a tapered feature that fixes the orthopedic plate 102 to the bone 101. The stationary pins 104 may be used to secure the orthopedic plate 102 to the first bone segment 101a in addition to one or more orthopedic screws or as an alternative to using orthopedic screws. Accordingly, one or more orthopedic screws and/or stationary pins 104 can be used to couple the orthopedic plate 102 to the first bone segment 101a in a fixed state.

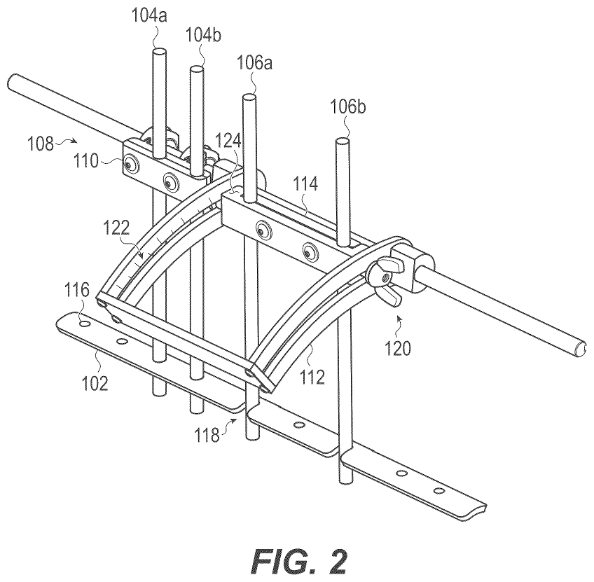

One or more movable pins 106 can be coupled to the bone 101 at a second side of the planned fracture location 103 (e.g., the second bone segment 101b) prior to cutting the bone. The end of the movable pin(s) 106 can include threads or any other suitable configuration to fixedly secure the movable pins 106 to the second bone segment 101b. In some cases, the orthopedic plate may include one or more recesses (shown as recess 118 in FIG. 2) and each movable pin 106 can be positioned at least partially within a recesses.

The rotation mechanism 108 can be coupled to the stationary pins 104 and the movable pins 106. The first body component 110 can be coupled to the one or more stationary pins 104 and include a guide 112 that defines a movement path of the rotation mechanism 108. The second body component 114 can be coupled to the movable pins 106 and the first body component 110. The second body component 114 can be configured to move along the guide 112 in the defined movement path.

After the rotation mechanism 208 is coupled to the stationary pins 104 and the movable pins 106, the bone can be cut at the fracture location 103. In some cases, the bone may be partially cut prior to coupling the orthopedic plate 102 to the bone. In these cases, the first bone segment 101a can be separated from the second bone segment 101b by completing the cut. After the bone 101 has been cut at the fracture location 103, movement of the second body component 114 causes the second bone segment 101b to move with respect to the first bone segment 101a.

FIG. 1B shows an example of the orthopedic fixation system 100 and the bone 101 in a rotated configuration after the bone 101 has been cut. The rotation mechanism 108 can define a movement path that causes the second bone segment 101b to rotate about an axis 105 that is offset from the rotation mechanism 208. For example, the guide 112 can define an arc and the curvature of the arc defines a location of the rotational axis. The orthopedic fixation system 100 can be configured to substantially locate the rotational axis of the rotation mechanism along the axis 105 the bone 101. For example, the rotation mechanism 108 can be positioned on the stationary pin(s) 104 and the movable pin(s) 106 that causes the second bone segment 101b to rotate axially with respect to the first bone segment 101a. Additionally or alternatively, the rotation mechanism 108 can be configured to be located outside the patient.

In some cases, the rotation of the second bone segment 101b can be performed by a surgeon moving a lower portion of a patient's leg (attached to the second bone segment 101b) while maintain an upper portion of the patient's leg (attached to the first bone segment) fixed. Accordingly, the orthopedic fixation system 100 may primarily limit the direction of motion to rotation of the second bone segment 101b to rotation about the axis 105 and the movement force is primarily applied to a portion of the patient's body (e.g., the lower leg). In other cases, the orthopedic fixation system 100 may be configured to also drive rotation of the second bone segment 101b. For example, the surgeon may grasp different portions of the orthopedic fixation system 100 to drive rotation of the second bone segment 101b with respect to the first bone segment 101a.

After a desired orientation of the first bone segment 101a and the second bone segment 101b has been set, the orthopedic plate 102 can be coupled to both the first bone segment 101a and the second bone segment 101b using any suitable techniques including orthopedic screws to lock the first and second bone segments 101a, 101b in a fixed orientation while the bone heals. After the orthopedic plate 102 has been coupled to the first and second bone segments 101a, 101b, the rotation mechanism 108, the movable pins 106 and the stationary pins 104 can be removed from the bone 101.

FIG. 2 shows the example orthopedic fixation system 100. The orthopedic plate 102 can include multiple holes 116 (one of which is labeled for clarity) and be secured to a bone using an orthopedic screw that engages with the holes 116.

As used herein, an orthopedic screw engaging with one or more holes 116 is meant to cover a variety of engagement mechanisms. In some cases, the orthopedic plate 102 can include threaded holes and threads on an orthopedic screw (e.g., threads on the head of an orthopedic screw) engage with the threaded holes. The threaded engagement of an orthopedic screw may fix the plate 102 with respect to the bone. In other cases, the orthopedic plate 102 can include unthreaded holes and a threaded body portion of an orthopedic screw may pass through the holes while a head of the orthopedic screw engages with the plate 102. In these cases, the primary fixing mechanism may come from compression of the plate against the bone by the orthopedic screw. In some cases, the plate 102 can have different types of engagement mechanisms, for example a combination of threaded and unthreaded holes.

In some cases, the orthopedic plate 102 can be configured to engage with locking orthopedic screws. As used herein the term “locking orthopedic screw” is used to define orthopedic screw that comprise features that lock a portion of the screw (e.g., the screw head) with respect to the plate as the screw is engaged with the plate 102. For example, the head of a locking screw can be configured to fix the plates 102 with respect to a bone.

The orthopedic plate 102 can include a number of holes 116 that allows one or more orthopedic screws to fix the plate to the first bone segment also include holes for one or more pins to pass through and be secured into the first bone segment. For example, if the orthopedic fixation system uses two stationary pins 104, the orthopedic plate 102 may include at least three holes 116 that can be positioned on a first side of a fracture location (e.g. over the first bone segment). The orthopedic plate 102 can also be configured with holes 116 that allows one or more orthopedic screws to fix the plate to the second bone segment. For example, if the orthopedic fixation system 100 uses two movable pins 106, the orthopedic plate 102 may include at least one hole 116 that can be positioned on a second side of the fracture location (e.g., over the second bone segment). However, the orthopedic plate 102 may include additional holes 116 that are positioned along the plate that can be used to fix the orthopedic plate 102 to the bone.

The orthopedic plate 102 can also include one or more recesses 118 (one of which is labeled for clarity). The recesses 118 can allow one or more movable pins 106 to be inserted into a bone and align with the stationary pins 104 while allowing the movable pins 106 to rotate or otherwise move without interfering with the orthopedic plate 102. The one or more recesses 118 can be positioned along a side of the orthopedic plate and can be positioned on a side that the movable pins 106 will be rotated away from. Different orthopedic fixation system 100 may have recesses 118 located on a side of the orthopedic plate 102 based on the direction of movement of the bone.

The rotation mechanism 108 can include a locking mechanism 120 that can selectively lock the second body component 114 to the first body component 110 to prevent movement between the first body component 110 and the second body component 114. When the locking mechanism 120 is locked it prevents movement of the second body component 114 along the guide 112 and the locking mechanism 120 is unlocked, the second body component 114 can be moved along the guide 112. The locking mechanism can include any suitable structures including locking nuts (e.g., wing nut), locking toggles or levers, and so on.

The orthopedic fixation system 100 can include a rotation indicator 122 that indicates an amount of angular rotation of the bone about the rotational axis (e.g., axis 105 shown in FIG. 1B). In some cases, the rotation indicator 122 can be positioned on the guide 112 and the positioning of the second body component 114 along the guide 112 can indicate the amount of angular rotation. For example, second body component 114 may include an indicator line 124 that aligns with markings on the rotation indicator 122 to provide the amount of angular rotation of the bone.

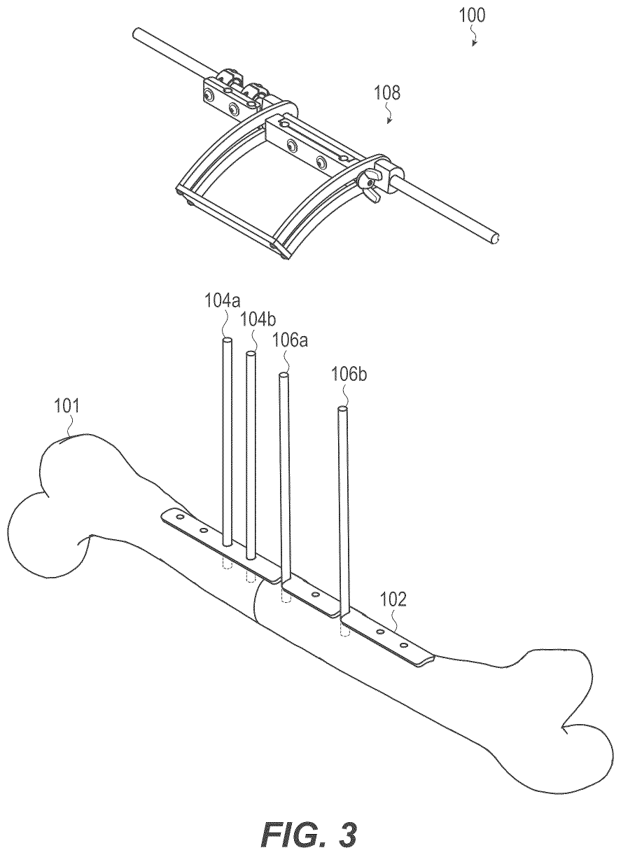

FIG. 3 shows the example orthopedic fixation system 100 with the rotation mechanism removed from the pins. During a surgical procedure, the orthopedic plate 102, the stationary pins 104 and the movable pins 106 can be coupled to the bone 101. The stationary pins 104 and the movable pins 106 can extend out of the patient. The rotation mechanism 108 can be coupled to the stationary pins 104 and the movable pins 106 and positioned external to the patient. The location of the rotation mechanism 108 on the pins can be adjusted to align the axis of rotation of the second body component 114 with an axis of the bone 101. After the desired positions of the rotation mechanism 108 on the stationary pins 104 and the movable pins 106 is set, the rotational mechanism can be fixed to the stationary and movable pins 104, 106 to prevent further movement between these components.

In some cases, the stationary and movable pins 104, 106 may be substantially aligned along the orthopedic plate 102. For example, the stationary and movable pins 104, 106 may be linearly aligned along a length of the orthopedic plate 102. In other cases, the stationary pins 104 and/or movable pins 106 can be initially set to the bone in other orientations. For example, a user may use an orthopedic plate that does not have one or more recesses such as found in many current orthopedic plates. Accordingly, the movable pins 106 can be coupled to the bone in a location that offsets the movable pins 106 to a side of the orthopedic plate 102. The rotation mechanism 108 can be adjusted to accommodate the offset of the moveable pins in the initial/unrotated configuration. Accordingly, the orthopedic fixation system 100 can be used with orthopedic plates that do not have a recces such as currently available orthopedic plates.

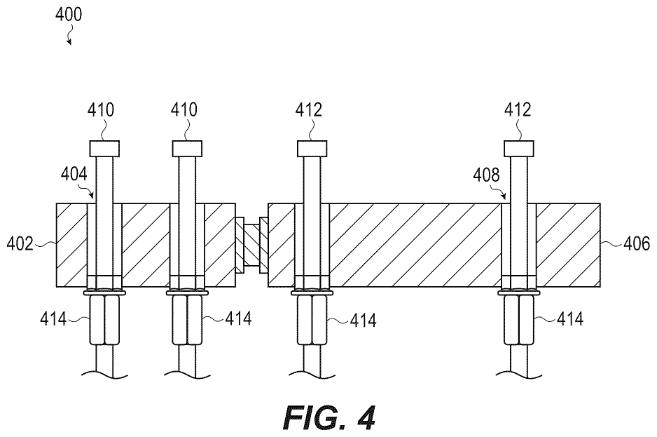

FIG. 4 shows an example a rotation mechanism 400 a set of pins. The rotation mechanism 400 can be an example of the rotation mechanisms described herein and include a first body component 402 and a second body component 406 that moves with respect to the first body component along a defined path. as described herein. The set of pins can include one or more stationary pins 410, which can be an example of the stationary pins described herein and one or more moveable pins 412, which can be an example of the movable pins described herein.

The first body component 402 can include one or more openings 404 that engage with the stationary pins 410 to couple the first body component 402 to the stationary pins 410. The second body component 406 can include one or more openings 408 that engage with the movable pins 412 to couple the second body component 406 to the movable pins 412. In some cases, the stationary pins 410 and/or the movable pins 412 can each include a stop 414 that can set the position of the rotation mechanism 400 with respect to the stationary and movable pins 410, 412. The openings 404, 408 may be configured so that a body portion of the stationary and movable pins 410, 412 can pass through the openings 404, 408. The stops 414 can prevent movement of the rotation mechanism 400 along the pins.

In some cases, the stops may include features that retain the rotation mechanism 400 on the pins 410, 412, for example to prevent movement of the rotation mechanism with respect to the pins 410, 412 during a procedure. For Example, the stops 414 can press fit into the openings 404, 408 to create a friction or interference fit that reduces the chance of the rotation mechanism 400 moving on the pins 410, 412. In some cases, the stops 414 can include features that prevent rotation or other movement of the pins 410, 412 with respect to the rotation mechanism 400. For example, the stops 414 can include one or more straight sides (e.g., a hexagon structure) to prevent rotation movement. Additionally or alternatively, the stops 414 and/or the rotation mechanism 400 can include other coupling features, such as a ball detent mechanism, snap mechanism or any other suitable structure to lock the rotation mechanism 400 to the pins 410, 412.

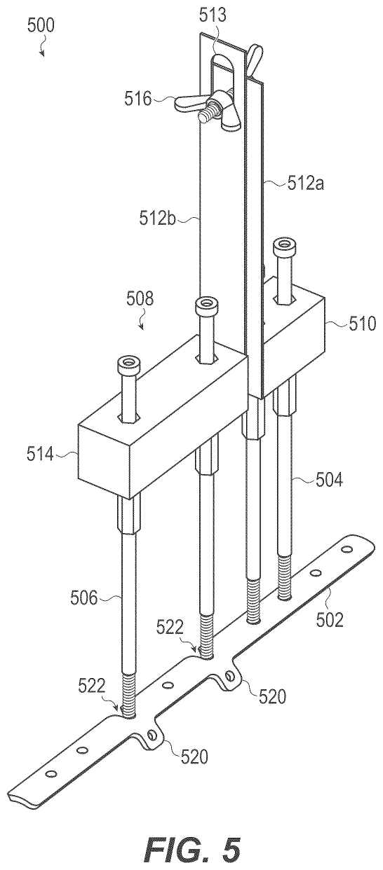

FIG. 5 shows an example orthopedic fixation system 500, which can be an example of the orthopedic fixation systems described herein. The orthopedic fixation system 500 can include an orthopedic plate 502, one or more stationary pins 504 (one of which is labeled for clarity), one or more movable pins 506 (one of which is labeled for clarity) and a rotation mechanism 508. The rotation mechanism 508 can include a first body component 510, a guide 512 and a second body component 514. In some cases, the rotation mechanism 508 can also include a locking mechanism 516.

The rotation mechanism 508 can be coupled to a bone using the stationary pins 504 and the movable pins 506 as described herein. The rotation mechanism 508 can include a guide 512 that controls the movement of the second body component 514 with respect to the first body component 510. A first guide tower 512a can be coupled to the first body component 510 and a second guide tower 512b can be coupled to the second body component 514. The second guide tower 512b can include a channel 513 that controls movement of the second body component 514. The channel 513 can allow the second body component 514 to rotate about a bone axis (e.g., axis 105) while the first body component remains stationary. In some cases, the guide 512 can also include structures that further define a movement path of the second body component 514 relative to the first body component 510. For example the second guide tower 512b can include a pins (not shown) that runs along a channel (not shown) in the first guide tower 512a. The pin and channel may define a radial path of the second body component 114 that contains motion of the second body component to rotation about the bone axis.

In some cases, the rotation mechanism 508 can include a lock mechanism 516, which can be selectively engaged to prevent movement between the first body component 510 and the second body component 514.

The orthopedic plate 502 can include one or more recesses 522 that allow the movable pins 506 to rotate (or otherwise move) with respect to the orthopedic plate 502, as described herein. The orthopedic plate 502 can also define one or more projections 520, which may include openings that an orthopedic fastener can be inserted into to couple the orthopedic plate 502 to bone. In some cases, each projection 520 is aligned with a recess 522 to maintain structural integrity of the orthopedic plate 502. The opening in the projection 520 may be also aligned with the recess 522 (as shown in FIG. 5) or offset from the recess 520. For example, the projection 522 may extend wider than the recess 522 and a corresponding opening may be offset from the recess 522 to prevent a pin (e.g., pin 506) and orthopedic fastener, inserted through the opening, from traveling through overlapping paths in the bone.

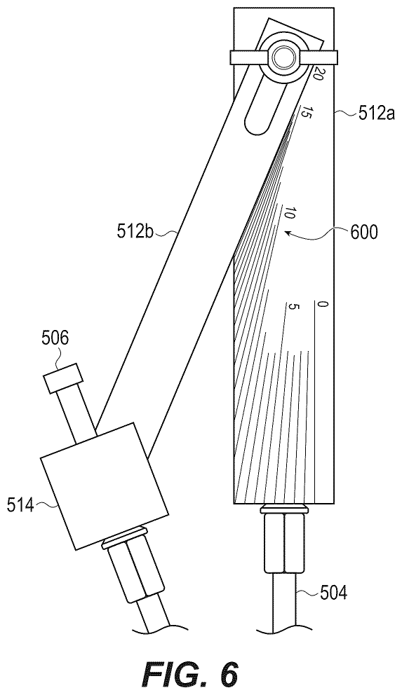

FIG. 6 shows an example rotation indicator 600 for the orthopedic fixation system 500 shown in FIG. 5. The rotation indicator 600 can include a set of marking positioned on the first guide tower 512a. As the second body component 514 moves along its defined path as the bone is rotated, the second guide tower 512b can move along the markings of the rotation indicator 600. The position of the second guide tower 512b (e.g., edge of the first tower) can be used to determine an angular rotation of the bone. In some cases, the movement of the guide 512 components may include both rotational movements and translational movements due to the structure and location of the guide 512 with respect to the axis of rotation of the bone. Accordingly, the markings of the rotation indicator 600 can be configured to account for the differences in movement of the guide 512 with respect to rotation of the bone and indicate an amount of rotation of the bone. For example, in some cases, the markings on the guide may be non-linear to account for both the translation and rotation of the second guide tower 512b with respect to the first tower 512a.

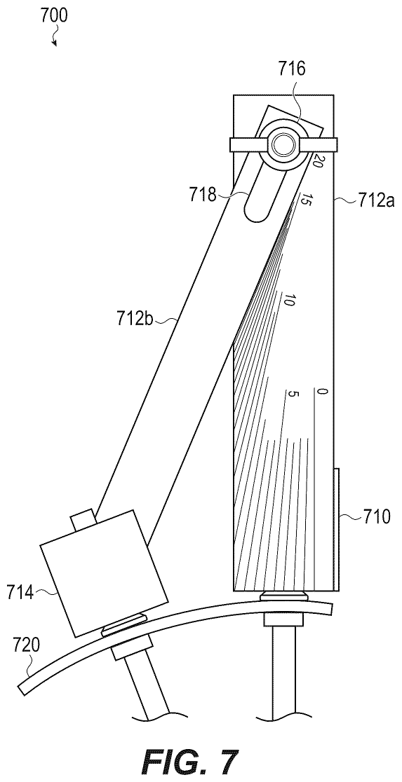

FIG. 7 shows an example orthopedic fixation system 700, which can be an example of the orthopedic fixation systems described herein. The orthopedic fixation system 700 can include a first body component 710 and a second body component 714 as described herein. In the example shown in FIG. 7, the orthopedic fixation system 700 includes a guide structure 712 that defines an angular movement path of the second body component 714 about the bone axis.

The first tower 712a can have a pin 716 and a second tower 712b can have an elongated opening 718 that moves along the pin 716. The first tower 712a can be coupled to the first body component 710 and the second tower 712b can be coupled to the second body component 714. The movement path defined by the pin 716 and the elongated opening 718 may allow for both rotation and translation of the second body component around a bone axis. In some cases, the orthopedic fixations system 700 can further include a rotational guide 720 that is coupled to the first body component 710 and defines a movement path for the second body component 714 that is constrained to rotation about a bone axis. For example, the second body component 714 may move along channels in the rotational guide 720 which limit movement of the second body component 714 to rotation about an axis defined by the rotational guide. The rotational axis defined by the rotational guide 720 can be configured to align or substantially align with an axis of the bone.

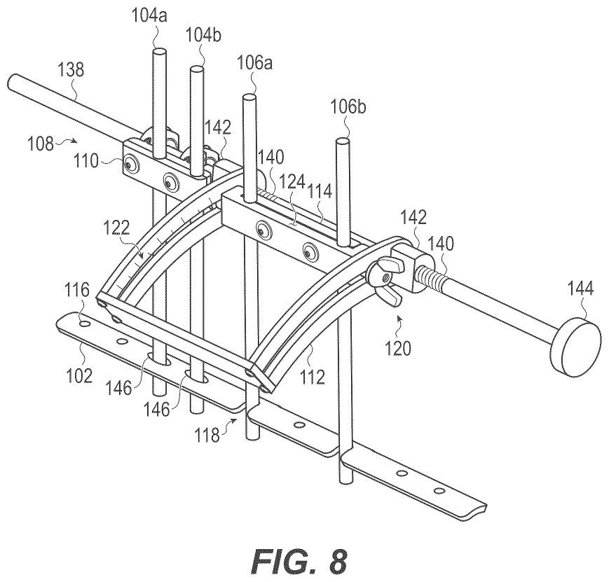

FIG. 8 shows an orthopedic fixation system 800, which may be an example of the orthopedic fixation systems described herein, such as orthopedic fixation system 100. The orthopedic fixation system 800 can include an orthopedic plate 102, one or more stationary pins 104, one or more movable pins 106, and a rotation mechanism 108 comprising a first body component 110 and a second body component 114, as described herein.

The orthopedic fixation system 800 can be configured to compress bone segments together and/or distract/separate bone segments. In some cases, the orthopedic fixation system 800 can include a rod 138 (or other structure or assembly) that couples the first body component 110 to the second body component 114. The rod 138 can be operated to change a distance between the first body component 110 and the second body component 114, to either compress bone segments or distract bone segments.

In some cases, the rod 138 includes one or more first sets of threaded features 140 and the second body component 114 can include one or more second sets of threaded features 142. The first set(s) of threaded features 140 can engage with the second set(s) of threaded features 142, and rotation of the rod 138 causes the rod 138 to move along an axial direction. The first component can be coupled to the rod 138 to allow rotation of the rod 138 with respect to the first component and prevent movement between the rod 138 and the first component 110 in other directions. For example, the first component 110 may include one or more bearings that couple the first component 110 to the rod 138. Accordingly, rotation of the rod 138 causes both the rod 138 and the first component 110 to move with respect to the second component 114. Rotation of the rod 138 (e.g., via knob 144) in a first direction can cause the first component 110 and the second component 114 to move closer together thereby com pressing bone segments attached to each of the first component 110 and the second component 114, as described herein. Rotation of the rod 138 in a second direction (opposite the first direction) can cause the first component 110 and the second component 114 to mover further apart from each other thereby distracting bone segments attached to each of the first component 110 and the second component 114.

In some cases, the orthopedic plate 102 includes elongated openings 146 that allow the stationary pins 104 to move as the rod changes a distance between the first component 110 and the second component 114. Additionally or alternatively, the orthopedic plate 102 can include elongated openings at the one or more movable pins 106 to allow compression or distraction of attached bone segments.

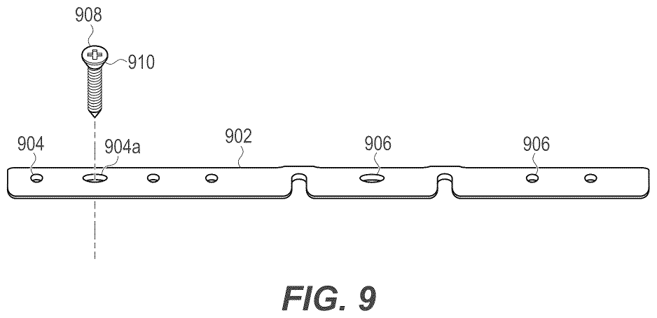

FIG. 9 shows an example of an orthopedic plate 902 that can be used with the orthopedic fixation systems described herein. The orthopedic plate 902 can be an example of the orthopedic plates described herein, including orthopedic plate 102. In some cases, openings in the orthopedic plate 902 can be configured to cause compression and/or extraction of two or more bone segments. For example, the orthopedic plate 902 can define one or more first openings 904 that are used to couple the orthopedic plate 902 to a first bone segment. The orthopedic plate 902 can also include one or more second openings 906 that are used to couple the orthopedic plate 902 to a second bone segment. One or more of the first openings 904 can include an oval shaped opening 904a. An orthopedic fastener 908 may include a tapered segment 910 that engages with the oval shaped opening 904a. Driving the orthopedic fastener 908 into a bone segment can cause the tapered segment 910 to contact an edge of the oval shaped opening 904a and cause axial movement of the attached bone segment. The oval shaped opening 904a and the orthopedic screw 908 can cause compression of a first bone segment attached to the orthopedic plate 102 by the orthopedic screw with a second bone segment attached to the orthopedic plate using one or more orthopedic fasteners engaged with one or more second openings 906.

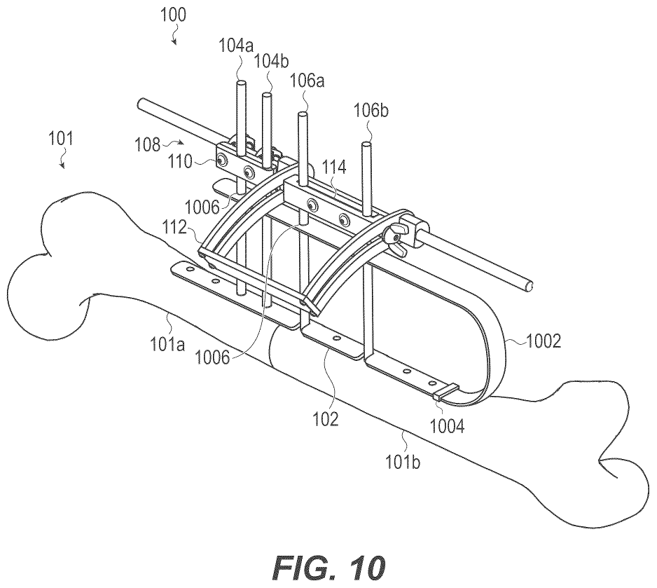

FIG. 10 shows an example of an orthopedic fixation system 100 including an alignment tool 1002 for use with an orthopedic plate 102. The alignment tool 1002 can be used during an orthopedic procedure to position the orthopedic plate 102 at one or more bone segments and align one or more of the fixed pins 104 and/or movable pings 106 with openings in the orthopedic plate 102 while the plate is positioned within a patient. The alignment tool 1002 includes a first end 1004 that removably couples the alignment tool 1002 to the orthopedic plate 1002. For example, the first end 1004 may define an opening and the orthopedic plate may be inserted into the opening and retained by frictional forces and/or other mechanisms (e.g., magnet, locking actuator, etc.). The alignment tool 1002 can also include one or more openings 1006 that are positioned outside a body when the orthopedic plate 102 and the first end 1004 are positioned within a body of a patient. The one or more openings 1006 may each align with a different opening one the orthopedic plate 102. Inserting a pin (e.g., a stationary pin 104 and/or movable pin 106) aligns the respective pin with an opening in the orthopedic plate 102. After one or more pins 1004, 1006 and/or other orthopedic fasteners have been engaged with the orthopedic plate 102, the alignment tool 1002 can be detached from the orthopedic plate 102 and removed from the user.

Additionally or alternatively, the alignment tool 102 can be used to locate one or more openings in the orthopedic plate while the plate is attached to one or more bone segments. For example, at a later procedure the alignment tool 1002 can be inserted into a patient and attached to the plate 102, which is also attached to one or more bone segments. Attaching the alignment tool 1002 to the plate causes the openings 1006 to be aligned with opening and/or orthopedic fasteners in the orthopedic plate 102. Accordingly, the alignment tool 102 can be used to locate orthopedic fasteners that are inserted into the plate (and bone) and/or locate one or more pins (e.g., pins 1004, 1006), which may be used to adjust the angular orientation of two bone segments, as described herein.

The foregoing description, for purposes of explanation, used specific nomenclature to provide a thorough understanding of the described embodiments. However, it will be apparent to one skilled in the art that the specific details are not required in order to practice the described embodiments. Thus, the foregoing descriptions of the specific embodiments described herein are presented for purposes of illustration and description. They are not targeted to be exhaustive or to limit the embodiments to the precise forms disclosed. It will be apparent to one of ordinary skill in the art that many modifications and variations are possible in view of the above teachings.

What is claimed is:

1. An orthopedic fixation system, comprising:
an orthopedic plate defining:
multiple openings each configured to engage with an orthopedic screw to secure the orthopedic plate to a bone; and
a recess positioned along a side of the orthopedic plate;
a set of pins comprising:
a first pin configured to pass through an opening of the multiple openings and be secured into the bone; and
a second pin that is positioned at least partially within the recess when the orthopedic plate is fixed to the bone and be secured into the bone; and
a rotation mechanism comprising:
a first body component that couples to the first pin and comprises:
a guide; and
a rotation indicator; and
a second body component that couples to the first body component and the second pin, the second body component configured move with respect to the first body component along the guide; wherein:
the guide causes the second body component to rotate substantially about an axis of the bone; and
the second body component indicates an amount of rotation on the rotation indicator.

2. The orthopedic fixation system of claim 1, wherein the guide and second body position cause the second pin to move out of the recess when the second body component is moved with respect to the first body component.

3. The orthopedic fixation system of claim 1, wherein:
the opening is configured to be positioned at a first portion of the bone located on a first side of a fracture site in the bone;
the recess is configured to be positioned at a second portion of the bone located on a second side of the fracture site in the bone; and
movement of the second body component along the guide is configured to cause the second portion of the bone to rotate with respect to the first portion of the bone.

4. The orthopedic fixation system of claim 1, wherein
the orthopedic plate defines a second recess positioned along the side of the orthopedic plate; and
the set of pins comprises:
a third pin that couples to the first body component an extends through a second opening of the multiple openings; and
a fourth pin that couples to the second body component and is positioned in the second recess.

5. The orthopedic fixation system of claim 1, wherein:
the second body component can move between an unrotated state and one or more rotated states; and
the first and second pins have a substantially linear alignment in the unrotated state.

6. The orthopedic fixation system of claim 1, wherein the guide prevents movement of the second body component in directions other than rotation about the axis.

7. The orthopedic fixation system of claim 1, further comprising a locking mechanism that is configured to:
in a first state, prevent movement between the first body component and the second body component; and
in a second state, allow the second body component to move with respect to the first body component and rotate about the axis.

8. The orthopedic fixation system of claim 1, wherein:
the first and second pins each comprises a stop that sets a position of the first and second body components along the first and second pins; and
the stop on each of the first and second pins positions the rotation mechanism outside a patient when the first and second pins are secured to the bone of the patient.

9. An orthopedic fixation system, comprising:
a first pin configured to be secured into a bone; and
a second pin configured to be secured into the bone; and
a rotation mechanism comprising:
a first body component that couples to the first pin and comprises a guide; and
a second body component that couples to the second pin, the second body component configured move along the guide, wherein the guide causes the second body component to substantially rotate about an axis of the bone.

10. The orthopedic fixation system of claim 9, wherein:
the first body component comprises a rotation indicator; and
the second body component indicates an amount of rotation on the rotation indicator.

11. The orthopedic fixation system of claim 9, further comprising and orthopedic plate, wherein:
the orthopedic plate defines multiple openings and a recess;
the first pin passes through an opening of the multiple openings then the orthopedic plate is positioned on the bone; and
the second pin is positioned at least partially within the recess when the orthopedic plate is positioned on the bone and the second body component is in an unrotated state.

12. The orthopedic fixation system of claim 11, wherein the second pin rotates away from the recess when the second body component moves along the guide from the unrotated state.

13. The orthopedic fixation system of claim 9, wherein the guide prevents movement of the second body component in directions other than rotation the axis of the bone.

14. The orthopedic fixation system of claim 9, further comprising:
a third pin configured to be secured into the bone and coupled to the first body component; and
a fourth pin configured to be secured into the bone and coupled to the second body component.

15. The orthopedic fixation system of claim 9 wherein:
the first body component is configured to couple to the first pin subsequent to the first pin being secured to the bone; and
the second body component is configured to couple to the second pin subsequent to the second pin being secured to the bone.

16. An orthopedic fixation system, comprising:
an orthopedic plate defining:
an opening; and
a recess positioned along a side of the orthopedic plate;
a first pin configured to pass through the opening and be secured into a bone; and
a second pin configured to be secured into the bone; and
a rotation mechanism comprising:
a first body component that couples to the first pin and comprises a guide; and
a second body component that couples to the second pin, the second body component configured to move along the guide, wherein the guide causes the second body component to substantially rotate about an axis of the bone.

17. The orthopedic fixation system of claim 16, wherein the guide substantially constrains motion of the second body component to rotation about the axis of the bone.

18. The orthopedic fixation system of claim 16, wherein:
the guide defines a radial path; and
the axis of the radial path substantially aligns with the axis of the bone.

19. The orthopedic fixation system of claim 16, wherein:
the first body component comprises a rotation indicator; and
movement of the second body component along the guide indicates an amount of rotation about the axis of the bone on the rotation indicator.

20. The orthopedic fixation system of claim 16, further comprising a locking mechanism that is configured to selectively lock the second body component to the first body component.
